(19) United States
(12) Patent Application Publication
(10) Pub. No.: US 2024/0157062 A1
(43) Pub. Date: May 16, 2024

(54) INJECTION SYSTEM HAVING SIMPLIFIED DATA TRANSMISSION

(71) Applicant: Ypsomed AG, Burgdorf (CH)

(72) Inventors: Leos Urbanek, Bern (CH); Silas Maechler, Aarberg (CH); Stefan Mangold, Liebefeld (CH); Peter Stettler, Ersigen (CH); Urs Kloetzli, Oberburg (CH); Adrian Eich, Wangenried (CH); Vinzenz Frauchiger, Solothurn (CH); Lorenz Schmid, Solothurn (CH)

(21) Appl. No.: 18/423,786

(22) Filed: Jan. 26, 2024

Related U.S. Application Data
(63) Continuation of application No. PCT/EP2022/071223, filed on Jul. 28, 2022.

(30) Foreign Application Priority Data
Aug. 2, 2021 (EP) 21189122.1
May 12, 2022 (CH) 000570/2022

(51) Int. Cl. A61M 5/50; G16H 20/17; G16H 40/67; H04L 9/40; H04W 12/03

(52) U.S. Cl. CPC A61M 5/5086; G16H 20/17; G16H 40/67; H04L 63/0442; H04W 12/03; A61M 2205/3327; A61M 2205/3553; A61M 2205/3584; A61M 2205/60

(57) ABSTRACT

The invention relates to an injection system in which drug delivery data are transmitted from a drug delivery device to a mobile device via a wireless Bluetooth connection and, from there, to a remote server via a network connection. A novel method makes it possible for the drug delivery data to be encrypted on the drug delivery device and to be sent via the Bluetooth transmission channel without pairing being carried out. The drug delivery data are decrypted only on the remote server and assigned to the drug delivery device. This process can be supported by both the mobile device and the drug delivery device observing the Bluetooth transmission channel and estimating how far away the relevant sender of the transmitted data is from the mobile device or drug delivery device.

### CROSS-REFERENCE TO RELATED APPLICATIONS

This application is a continuation of International Application No. PCT/EP2022/071223, filed Jul. 28, 2022, which in turn claims priority to European Application No. 21189122.1, filed on Aug. 2, 2021 and Swiss Application No. CH000570/2022, filed on May 12, 2022, each of which is incorporated by reference herein, in the entirety and for all purposes.

### TECHNICAL FIELD

The present invention relates to the field of medical injection systems, such as smart injection systems, which can transmit data from a drug delivery device for medication to other devices and which can optionally be easily adapted to the needs of various medical indications if required.

### BACKGROUND

More and more diseases and illnesses are becoming treatable. Increasingly expensive medications and treatments are finding their way into therapy. Therefore, controlling costs while ensuring patient safety and data protection is becoming increasingly important. On the one hand, attempts are being made to enable home therapies even for more complex therapies, which can reduce healthcare costs. On the other hand, for example in the case of more expensive therapies, it should be possible to ensure that medications are used correctly by patients, even in the case of home therapy, and that patient compliance is ensured.

Injection systems are known from the prior art in which drug delivery devices send information about the use of the drug delivery devices to a mobile device via Bluetooth, and the mobile device forwards at least some of the information to one or more remote servers. Since this information may comprise sensitive patient information, this information may be transmitted securely to protect it from unauthorized access. The connection between the mobile device and the drug delivery device may thus be protected, as well as the connection between the mobile device and remote server computers. In addition, the information may also be stored securely on the respective devices and computers.

An encrypted Bluetooth connection is typically used to connect the mobile device and the drug delivery device. For this purpose, the mobile device and the drug delivery device may go through a coupling process, which, among other things, enables unique identification and assignment among the communication partners. In the Bluetooth communication protocol, this coupling is called “pairing”. To further secure the connection, further encryption can be established at the application level between the mobile device and the drug delivery device. This multi-level security may often require a coupling, pairing and key exchange process initiated by a user. For simpler injection devices such as autoinjectors, securing the connection between the injection device and the mobile device can thereby take more time than the actual injection process. For patients, multi-level security of the connection is also associated with the risk of incorrect handling, for example in the case of visual impairment. There is therefore a need for easy-to-use and yet secure information transmission for injection devices which, for example, are only used for one injection and are disposed of after use.

Manufacturers of injection apparatuses, medication manufacturers, health insurers and people requiring care may also need to have injection apparatuses to hand that are as safe and effective as possible. Accordingly, it would be desirable to have an injection apparatus which has a consistent, proven, safe and predetermined technical design having a consistent housing, where the outer shape, feel, surface topography and ergonomics of the injection apparatus can be tailored even during production of the device using additional attachments that are firmly and permanently connected to the housing, which allows the needs of the medical indication to be taken into account in a simple and cost-effective manner.

### SUMMARY

It is an object of the invention to provide alternative injection systems which allow for simplified and secure transmission of injection data from an injection device to a remote server.

The object is achieved by a method and a system according to the implementation of disclosure.

In one embodiment of the invention, the invention includes a system for the secure transmission of medical information. The information is first transmitted from a drug delivery device to a mobile device and then from the mobile device to a remote server or computer.

The drug delivery device can be a patch injector, an autoinjector, an injection pen or an infusion pump. Further possible drug delivery devices are devices for inhaling substances via the respiratory tract, both for medical purposes and recreational use. According to the invention, drug delivery devices can also be constructed modularly from components which can or cannot be detached from one another. Typical modules are a reservoir unit having the reservoir for disposable use, a communication module and a reusable drive unit having the drive. A patch injector and an autoinjector are used as examples below for the description of the invention.

Such an autoinjector includes a device for administering fluid medication, as is known to a person skilled in the art from commercially available autoinjectors. Furthermore, the autoinjector includes an electronic controller. The controller can be connected to sensors which can also be provided in the autoinjector and can receive and process the measurement signals from the sensors. Furthermore, the controller is connected to a memory provided in the autoinjector, in which memory the controller can store data. A Bluetooth unit is arranged in or on the autoinjector, which Bluetooth unit is connected to the controller and via which data can be sent and received wirelessly. The Bluetooth unit can send and receive data in a transmission or broadcast mode in the form of advertising packets described above, i.e., without an identifier, address or other previously known identification for one or more receiving devices. In one embodiment, the Bluetooth unit functions exclusively as a beacon in transmission or broadcast mode. In an autoinjector equipped in this way, drug delivery can be monitored via the sensors. The controller can receive the sensor signals, store them and/or distribute them wirelessly via the Bluetooth unit.

In an alternative and optional form of a drug delivery device, the drug delivery device also includes, in addition to the elements obvious to a person skilled in the art, a plurality of housing-like shells, which may be attached to the outside of the housing of the drug delivery device. Drug delivery devices, such as autoinjectors, having the housing-like shells mentioned are also known from the prior art, such as in WO 2016179713 A1. What is special about this form is that at least some of the electronic elements described above for the autoinjector are arranged on the housing-like shells. The housing-like shells enable the external appearance of the drug delivery device to be adapted in terms of shape, material, color, ergonomics and/or feel to the requirements of the medical indication.

According to the invention, the system includes a mobile device. This can be a smartphone, a PDA, a tablet, a notebook or a similarly suitable device. According to the invention, the mobile device also contains a Bluetooth unit and a network unit for communication with remote devices. This can be an Ethernet unit, i.e., a wired network unit, a wireless network unit for a WLAN or similar system, a mobile communication unit or even a cell phone unit of any generation. Both Bluetooth and network are common functionalities in the device classes mentioned, are widespread and well known to a person skilled in the art.

According to the invention, the system includes at least one further remote computer or server. The at least one computer does not necessarily have to be a real computer, but can also mean one or more cloud instances, i.e., virtual computers or containers. The at least one computer or server computer includes at least one, possibly virtual, processor, a memory and a connection to a network. These components are common components in virtual and real servers and do not require any further explanation for understanding the invention.

The autoinjector, as a non-limiting example of an injection or infusion device according to the invention, has, as mentioned, a memory in which the controller can store data. Furthermore, at least one cryptographic key is stored in this memory, with which the controller can encrypt or certify data if required and can either store them in encrypted form in the memory or can distribute them in encrypted form via the Bluetooth unit of the autoinjector. An identifier for the autoinjector is then also stored in the memory. In a drug delivery device of modular design, the memory can also be constructed of several parts and distributed across the various modules. The key can be stored in a memory in a reservoir unit in order to ensure assignment to the medication.

The controller of the autoinjector can now encrypt drug delivery data which, for example, have been recorded and stored by means of the sensors, and, at least together with the identifier for the autoinjector, distribute them as an advertising packet via a Bluetooth unit in transmission or broadcast mode, for example as a sequence of several packets in a row. The identifier for the autoinjector is unique and is ideally only allocated once. In some embodiments, the drug delivery data may also comprise an absolute or relative time and/or date stamp. Furthermore, the drug delivery data may contain temperatures, sensor data, time measurements, states or activities.

The mobile device can receive the transmission in the form of the advertising packet via its own Bluetooth unit. For example, a corresponding app is installed on the mobile device which receives the data. However, the app does not have a key that would enable decryption of the encrypted data. On the other hand, it recognizes the identifier for the autoinjector and, if the autoinjector sends several packets, can classify these correctly. The app can then forward the at least one advertising packet received via the network unit of the mobile device to a computer or server known to the app.

The remote computer or server computer, hereinafter referred to as the server, can receive the transmission forwarded by the mobile device via its network connection. The device identifier can be read on the server. In one embodiment, a database in which the identifiers of the devices are stored is stored on the server. This database also contains a matching key for each identifier, with which key the encrypted data on the server can be decrypted. In an alternative embodiment, the server can have access to a further server on which the database is stored.

The decrypted data can then be used to analyze the drug delivery data, or, for example, to check when the autoinjector was used or whether a storage temperature was exceeded.

In one embodiment of the invention, the key in the drug delivery device for encrypting the data and the key on the remote computer or server form an asymmetric key pair. This corresponds to the common asymmetric key pairs of public and private keys, as are known to a person skilled in the art from the Internet. This has the advantage that data which have been encrypted with the key on the drug delivery device can only be decrypted with the key on the server. Even if a potential attacker could extract a key from the drug delivery device, they would not be able to access already encrypted data. Alternatively, the key pair can also be symmetrical, which also works when accepting the aforementioned disadvantage.

Since the mobile device does not store any of the keys, it does not have access to the actual drug delivery data either. If desired, decrypted data to be provided to a patient could be processed by the server and sent back to the app. The data can be evaluated and prepared on the server beforehand so that they may be provided in an easy-to-understand manner on the app.

In an embodiment, no Bluetooth pairing takes place between the drug delivery device and the mobile device before, during or after data transmission. In a further development, it is desirable that the Bluetooth unit of the drug delivery device is not capable of pairing at all, but is electronically reduced to the essentials, namely the ability to send or receive advertising packets.

In yet another development, the reception possibility is also omitted. This reduction to the essentials saves components and reduces potential sources of error.

In a further embodiment according to the invention, the drug delivery device according to the invention is of modularly design. For example, the drug delivery device can be formed from two modules, which can be detachably connected to one another. One module can be an injection or infusion module and the other can be a communication module. The communication module may contain electronic sensors, a controller, at least one processor, at least one memory component and a Bluetooth unit. The injection or infusion module could be designed as a classic injection pen, as an autoinjector or as a patch device without any electronics. This would, for example, allow the injection or infusion module to be designed as a disposable module and the communication module to be designed as a reusable module for repeated use. The modules described could each be integrated as components in a drug delivery device, or could be coupled to a drug delivery device as an additional module and could be detached therefrom again.

In a further embodiment according to the invention, after receiving an advertising packet from a drug delivery device, the mobile device also sends an advertising packet as a confirmation of receipt via the Bluetooth unit. In some embodiments, this confirmation of receipt is only sent when the mobile device has, in turn, received feedback from the server that the advertising packet that originally came from the drug delivery device has arrived at the server. Receipt of the packet is confirmed in the confirmation of receipt to the drug delivery device. The confirmation contains at least the identifier for the drug delivery device that sent the packet to be confirmed. Typically, a unique identifier for the mobile device is attached. As part of the packet length, other parameters such as a packet number can also be transmitted. The drug delivery device can receive and analyze or evaluate the confirmation of receipt.

The mobile device can receive advertising packets from a plurality of drug delivery devices and also confirm them. According to the invention, a drug delivery device can also receive advertising packets both from a mobile device and from a second drug delivery device.

If several drug delivery devices are now close to one another and can receive advertising packets from one another and confirmations of receipt from at least one mobile device, the drug delivery devices may use the additional information contained in the advertising packet or obtained therefrom upon receipt.

For example, a drug delivery device that receives such additional information can, the next time drug delivery data are transmitted, supplement (and encrypt) the drug delivery data with the information that additional drug delivery devices have been discovered in the vicinity. At most, the added information can include the identifier for the additional drug delivery device(s). Proximity, or the recognition of presence, can be meant qualitatively or quantitatively. Qualitatively means that a mobile device receives a signal from a drug delivery device. Quantitatively means that the mobile device can also estimate a measure of proximity. This can be done, for example, via the RSSI function, where RSSI means Received Signal Strength Indication (see the Bluetooth Core Specification, publicly available from Bluetooth SIG Inc., Kirkland, WA 98033, U.S.A., for example Version 5.2 dated 31 Dec. 2019).

If the server then receives such information, it can use the presence of additional evaluations. If the server also knows the other drug delivery devices (from the database), in one embodiment, the transmission reliability of the drug delivery devices can be recognized. Alternatively or additionally, a check can be made as to whether all of the recognized drug delivery devices, or the medications contained therein, are compatible with one another. If they are not, the server can send a warning to the app. In a further alternative, the server can also send recommendations to the app, for example recommending a specific order in which the various medications should be administered.

In further embodiments of the invention, a plurality of mobile devices may simultaneously receive information from a drug delivery device. In these embodiments, the Bluetooth units of the mobile devices may have implemented the RSSI (Received Signal Strength Indication) function (see the Bluetooth Core Specification, reference given above). This makes it possible for the mobile device to estimate the receiving distance, i.e., the distance between the transmitter and receiver at the time of data transmission, between the drug delivery device and itself. In this embodiment, the mobile device can attach the information extracted from the RSSI in this way to the advertising packet to be forwarded. Accordingly, identical advertising packets that were forwarded to the server via different mobile devices can be received on the server. For example, the server can then decide which of the identical advertising packets should be further processed. The information extracted from the RSSI function can help. For example, the server may decide that the advertising packet from a set of identical advertising packets, for which advertising packet the RSSI function indicates the smallest receiving distance between the mobile device and the drug delivery device, will be retained. The remaining advertising packets from the set of identical advertising packets can then be discarded, for example. Alternatively, all packets can also be used together for further evaluations. For example, it can be examined how many mobile devices are within the transmission range of a single drug delivery device which receive and forward the advertising packets. In this way, local clusters of patients being treated for the same disease may be identified.

### BRIEF DESCRIPTION OF THE DRAWINGS

Exemplary embodiments of the invention are described below in connection with the appended figures. These embodiments are intended to show basic possibilities of the invention and are in no way to be interpreted as limiting.

FIG. 1 shows a first system according to the invention consisting of a patch injector 1, a smartphone 13 and a server 14.

FIG. 2 shows a second system according to the invention consisting of an autoinjector 11 having a built-in electronic module 11a, a smartphone 13 and a server.

FIG. 3 shows a third system according to the invention consisting of a modular injection device (an autoinjector 11 having an add-on 12), a smartphone and a server.

FIG. 4 shows the flowchart of a method according to the invention for transmitting drug delivery data for dose administrations from a reservoir of a drug delivery device.

FIG. 5 shows a fourth system according to the invention in which a plurality of drug delivery devices communicate with a server via a mobile device.

FIG. 6 shows the flowchart of a method expanded according to the invention from FIG. 4 showing communication with two different mobile devices.

FIG. 7a-7e show an optional drug delivery device consisting of an injection device 802 and additional housing shells 830 and 830′. The shell 830′ includes the electronic module 840.

FIGS. 8a and 8b are detailed views of the electronic module 840.

### DETAILED DESCRIPTION

### Definitions

The term “product”, “medication” or “medical substance” in the present context includes any flowable or fluid medical formulation which is suitable for controlled administration by means of a cannula or hollow needle in subcutaneous or intramuscular tissue, for example a liquid, a solution, a gel or a fine suspension containing one or more active medical ingredients. A medicament can thus be a composition with a single active ingredient or a premixed or co-formulated composition with a plurality of active ingredients from a single container. The term includes drugs, such as peptides (e.g., insulins, insulin-containing medicaments, GLP 1-containing preparations as well as derived or analogous preparations), proteins and hormones, biologically obtained or active ingredients, active ingredients based on hormones or genes, nutrient formulations, enzymes, and other substances both in solid (suspended) or liquid form. The term also includes polysaccharides, vaccines, DNA or RNA or oligonucleotides, antibodies or parts of antibodies as well as suitable base substances, excipients, and carrier substances.

In the present description, the terms “injection system”, “injection device”, “injection apparatus” or “injector” are understood to mean an apparatus in which the injection needle is removed from the tissue after a controlled amount of the medical substance has been dispensed. In contrast to an infusion system, the injection needle in an injection system or in an injector thus does not remain in the tissue for a longer period of several hours. In addition to the actual injection device, an injection system may comprise one or more additional injection devices. Bluetooth is well known as a wireless technology. Bluetooth beacons are a little less well known. A beacon refers to a transmitter or receiver which is based on Bluetooth Low Energy BLE or Bluetooth Smart Technology BST. Basically, this is a wireless technology that can be understood as a development of Bluetooth. As with any wireless technology, at least one transmitter and at least one receiver are first required. The transmitters are referred to here as “beacons”. Their range is around ten meters, depending on the level of development and the manufacturer. Far less energy is used to transmit data between devices using BLE than with older versions of Bluetooth, which, for example, results in lower battery consumption for the transmitter. The costs of the transfer also remain low. Large amounts of data are not suitable for exchange because the transmission rate is relatively low. The beacons themselves are powered by batteries or, more rarely, a direct power supply. Beacons are already in use in many places to locate visitors in a store and send location-dependent offers to the respective terminals. For example, beacons are used at Hamburg Airport to measure the time spent in various stores and reward them with bonuses.

Bluetooth beacons can be used as transmitters for small amounts of data. The data are not transmitted in a point-to-point connection, but rather the Bluetooth beacon sends the data in transmission or broadcast mode, as a so-called advertising packet, which is received by all receiving Bluetooth devices that are within wireless communication range of the transmitting beacon. This means that receiving devices do not need to be paired with the beacon via Bluetooth pairing and/or identified in any way before data transmission. For example, in early versions of Bluetooth, an advertising packet may contain 31 bytes of data. The data can include, for example, a transmitter identification and other data. The term “reception” in this document does not necessarily include the interpretation of the data. This allows data packets having encrypted content to be received and forwarded without recognizing or interpreting the content. In telecommunications, it has always been well known to a person skilled in the art that certain information can be obtained from the reception of signals, regardless of the content of the communication, which information is useful for controlling the transmission. The simplest example of this is measuring the signal strength when receiving wireless signals, which is used in many communication systems to control antenna gain or transmission power.

For details about Bluetooth in general, Bluetooth Low Energy and beacons which use Bluetooth technology, please refer to the Bluetooth specification, which can be accessed online at the website of publicly available from Bluetooth SIG Inc., Kirkland, WA 98033, U.S.A. at https://www.bluetooth.com/specifications/specs.

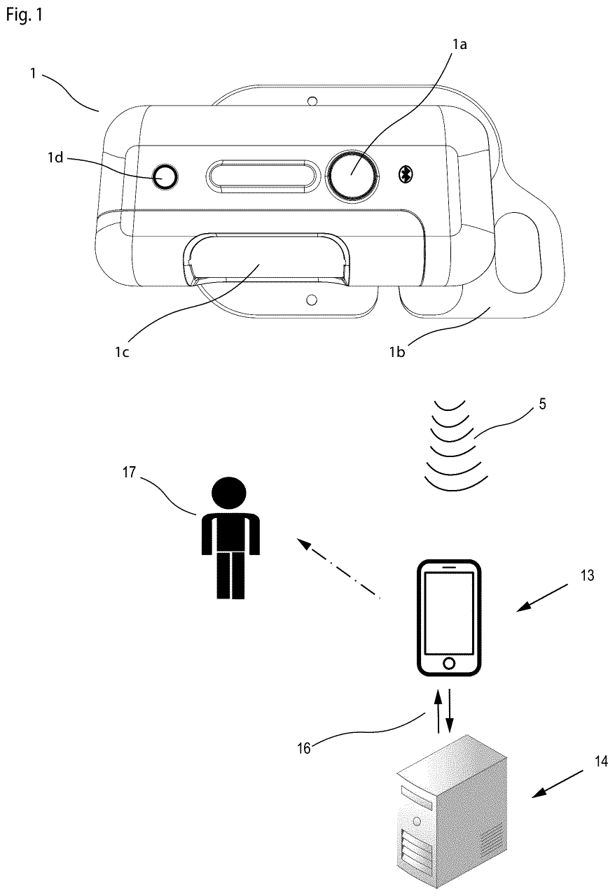

FIG. 1 shows a first system according to the invention. The drug delivery device according to the invention is an electronically operated patch injector 1. Patch injectors are injection devices which are attached directly to the patient's skin by means of a patch. After attaching the injector 1, the injection of the fluid medication can be started by pressing the start button 1a. For example, the status LED 1d shows the current status of the injector 1 by means of a color code in this case. Furthermore, the progress of the injection can also be observed through the viewing window 1c, through which the medication reservoir is visible. The injector includes further sensors which can determine the various states of the injector. For example, a capacitive sensor can be arranged in the patch (not visible in FIG. 1) of the injector, which can be used to determine whether the injector adheres to the skin. Another sensor determines when the start button 1a is pressed and a further sensor determines the end of an injection (for example when a piston rod in a syringe/reservoir reaches a certain position). As mentioned, the drug delivery device 1 is an electronically operated injector. In addition to the electronic controller and various sensors, the injector 1 also contains a memory in which the drug delivery data can be stored, a unique identifier for the injector and a key for encrypting the drug delivery data. A Bluetooth unit is also integrated into the injector 1 and connected to the controller. Once activated (for example by removing the protective film 1b), the controller can now continuously query the sensors and send possible status changes as encrypted data together with the unique identifier as advertising packets by means of the Bluetooth unit via a transmission or broadcast channel 5. If there is no confirmation from the mobile device or smartphone 3, a packet can be sent repeatedly. The mobile device 3 can then send each of the advertising packets to the server 4 via the network connection 6. As described, the server 4 can decrypt and analyze or evaluate the data based on the information relating to the identifier and the matching key available in a database. Alternatively, the electronics required for control and communication purposes can be integrated into the patch injector in the form of an electronic module 11a.

An embodiment of the patch injector 1 obvious to a person skilled in the art is a modular drug delivery device 1, with a reservoir unit including the disposable reservoir, a reusable drive unit including the drive, and a memory provided in the reservoir unit to store the key.

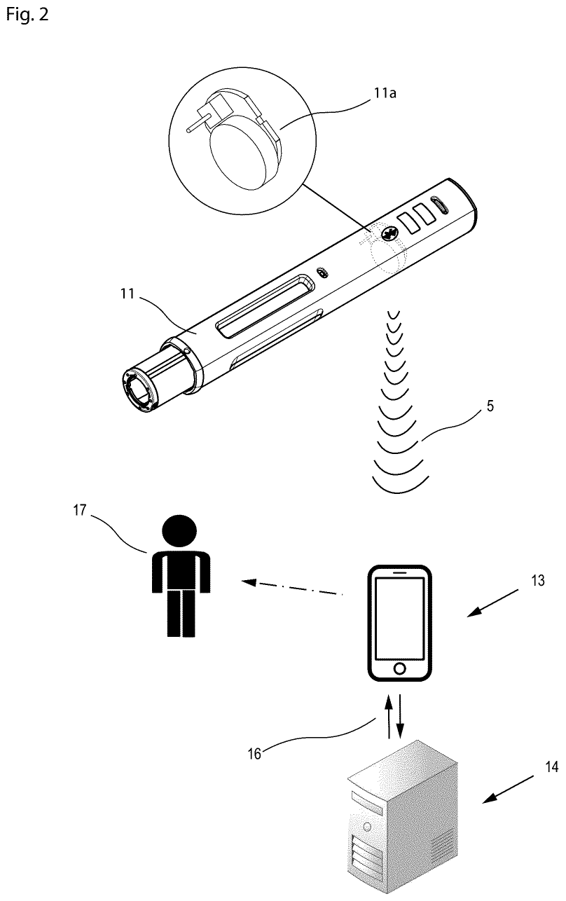

FIG. 2 shows a drug delivery device in the form of an autoinjector 11 having an integrated electronic module 11a.

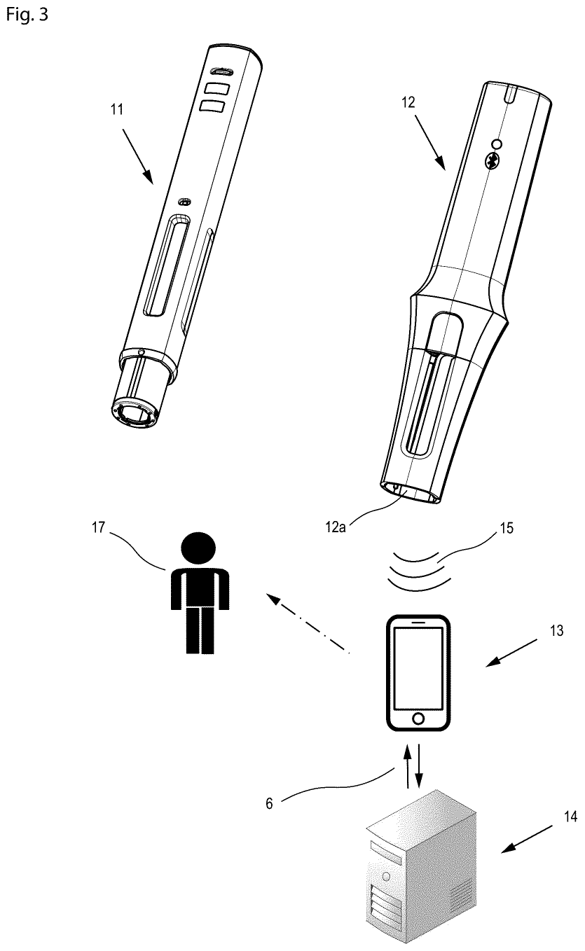

FIG. 3 shows a second system according to the invention. In this embodiment, the system is of modular design and consists of an autoinjector 11 without an electronic module, and an additional module 12. The autoinjector 11 can be pushed by its rear end into the opening 12a in the add-on 12. The two parts can be snapped together to create a unit. The add-on 12 includes the elements according to the invention of the electronic module 11a having a Bluetooth unit, and can read the key from an NFC memory of the autoinjector in order to uniquely identify the reservoir. With regard to the invention, the unit consisting of the add-on 12 and the autoinjector 11 functions in the same way as the system from FIG. 1. Encrypted data are distributed wirelessly together with the identifier in advertising packets via a Bluetooth transmission or broadcast channel 5. The mobile device, here also a smartphone 3 in FIG. 3, forwards the packets to the server 4 via the network 6.

Evaluated data can only be sent back to the smartphone 3 from the server 4 via the network 6, which data can then be viewed by the person 7 on the smartphone.

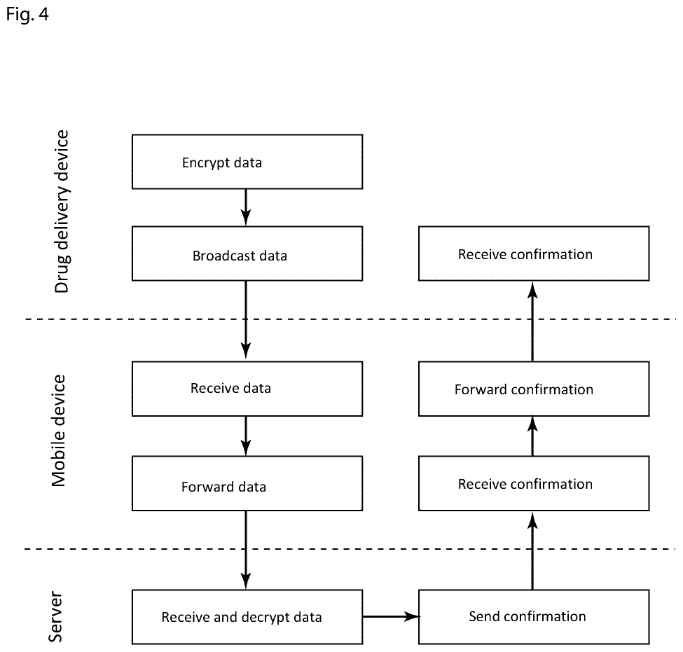

FIG. 4 shows a flowchart for a method according to the invention for transmitting drug delivery data for dose administrations from a reservoir of a drug delivery device 1, comprising the following steps:
Encrypting the drug delivery data with the aid of a key on a processor of the drug delivery device 1. Drug delivery data may include information regarding the quality, success, medicine volume, time or duration of an injection or sub-steps of an operating sequence carried out on the drug delivery device 1 for the purpose of administration. The key stored on the drug delivery device 1 may, for example, form an asymmetric key pair with a second key stored in a database on the server computer 4. The distribution of the keys is selected such that the data encrypted on the drug delivery device 1 can only be decrypted with the matching key of the server computer 4. Other devices involved in the communication or storage of the drug delivery data, such as the mobile device 3, may typically not have the matching key.
Wirelessly transmitting or broadcasting the encrypted drug delivery data together with a unique identifier for the drug delivery device 1 in transmission or broadcast mode as transmission or broadcast data via a Bluetooth unit of the drug delivery device 1 and a Bluetooth broadcast channel 5. These are typically BLE advertisement frames having data packets without specifying the recipient, so that previous connection (pairing) is not necessary.
Receiving the transmission or broadcast data, the unique identifier and possible further components of a data packet via a Bluetooth unit on a mobile device 3. Because the data can be transmitted without prior establishment of a connection, this also includes ongoing tracking of the wireless data traffic in the vicinity of the mobile device 3, as well as an analysis thereof, so that drug delivery data from a drug delivery device 1 are recognized as such. It is also possible to include the user 7 in the process and, for example, to request a manual confirmation. Decrypting data is not necessary for this purpose.
Forwarding the transmission or broadcast data, such as the encrypted drug delivery data, from the mobile device 3 to a remote server computer 4 via a network interface of the mobile device 3. The server computer 4 may also be implemented on a plurality of processors or computers which work together for the purpose of processing drug delivery data. The mobile device 3 typically cannot decrypt the transmitted drug delivery data itself because, according to the invention, the key required for this is only available on the server computer 4.
Receiving and decrypting the drug delivery data encrypted in the transmission or broadcast data by the server computer 4 with the aid of a matching key stored on the server computer 4, which key is identified using the unique identifier. The keys required for operation can be provided on the server computer 4 during installation or commissioning of the system, for example.
Sending, by the server computer 4, a confirmation of receipt to the mobile device 3. The confirmation of receipt includes at least a unique identifier for the drug delivery device 1, but can also contain further data, such as the identifier for the mobile device 3, elements from the Bluetooth communication protocol or drug delivery data.
Receiving the confirmation of receipt by the mobile device 3 and forwarding it to the drug delivery device 1 via the Bluetooth broadcast channel 5. No connection (pairing) is, in turn, necessary for this. The mobile device 1 can also display the confirmation of receipt on a screen or bring it to the attention of the user 7 in another manner. Here, too, it is possible to involve the user 7 in the process and, for example, to request a manual confirmation, either with each data transmission or selectively on the basis of an evaluation of the transmission conditions.
Receiving the confirmation of receipt by the drug delivery device 1.

The feedback of a confirmation from the server 4 to the drug delivery device 1 (right side in the flowchart in FIG. 4) is not absolutely necessary, but may contribute to securing the transmission. In the same way, manual confirmation of data packets and control of communication by the user 7 can also be omitted or can be carried out at various points in the process described. In the process above, two positions are mentioned by way of example which may be suitable for securing data transmission.

An advantage of the present invention is that direct, encrypted communication between a drug delivery device and a mobile device is possible according to a point-to-point connection, without the devices involved having to first establish and authenticate such a direct connection before exchanging drug delivery data.

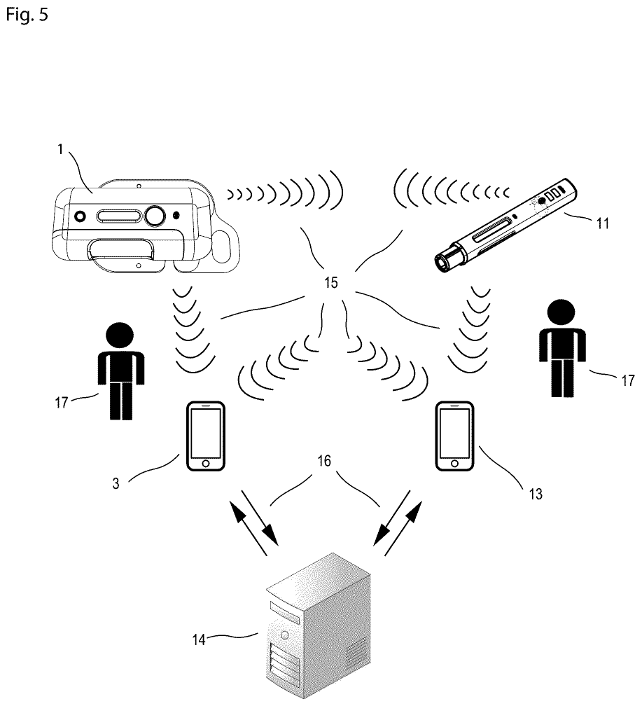

FIG. 5 shows a system according to the present invention, in which several drug delivery devices 1, 11 communicate with the server computer 4 via at least one mobile device 3a, 3b each time. Again, no explicit assignment (pairing) between the mobile device 3a, 3b and the drug delivery device 1, 11 is necessary for communication, which significantly reduces operational complexity. Such an assignment is very useful for securing the transmission of drug delivery data to the server 4, or for displaying device-specific information on the mobile device 3a, 3b. An extension of the process from FIG. 4 now allows the drug delivery devices 1, 11 within a Bluetooth communication range to support such an assignment. In FIG. 5, it can also be seen that drug delivery devices 1, 11 which are used at a sufficiently short distance from one another can also communicate with one another. Drug delivery data and all information sent by the drug delivery device 1 or mobile device 3a in transmission or broadcast mode can thus also be received by the drug delivery device 11 and can be used to secure the system, to support the assignment of the mobile device 3a, 3b to the drug delivery device 1, 11, and to implement further functions such as a graphical representation of the network topology. Similarly, both drug delivery devices 1, 11 can also receive confirmations from both mobile devices 3a, 3b and use them to implement the aforementioned functions. In such a way it may be possible for each drug delivery device 1, 11 to make an estimate for each data packet received via a Bluetooth broadcast channel 5 as to how great the receiving distance is for these data—i.e. the distance between the transmitter and the receiver—be it a mobile device 3a, 3b or another drug delivery device 1, 11. The evaluation of this information is another important aspect of the present invention. In the simplest case, the drug delivery device 1, 11 can issue a non-specific warning that a plurality of drug delivery devices 1, 11 or mobile devices 3a, 3b are in proximity.

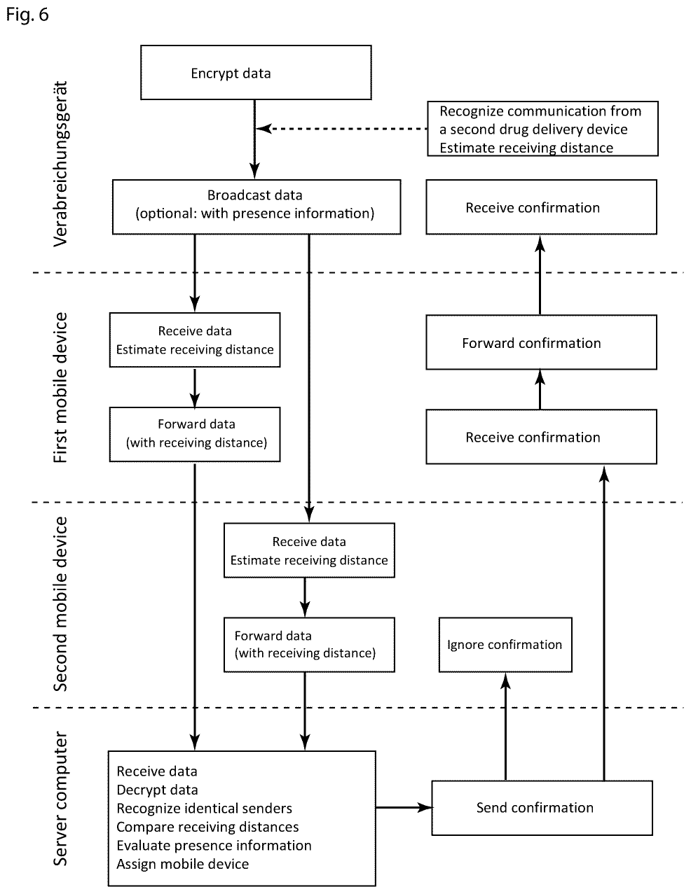

FIG. 6 shows an expanded flowchart which includes, in addition to the process described in FIG. 4, the following steps:
Receiving the broadcast data via a Bluetooth unit on a first mobile device 3a, estimating a first receiving distance between the first mobile device 3a and the first drug delivery device 1 based on the reception of the broadcast data, and forwarding the first receiving distance to the server computer 4. The receiving distance can be estimated by evaluating the Received Signal Strength Indication (RSSI) that is also sent according to the Bluetooth standard, or by evaluating other suitable information, such as Bit Error Rate (BER) or Quality of Service (QoS). A measure for the receiving distance is attached to or combined with the drug delivery data and the identifier for the drug delivery device 1 so that all three pieces of information are available to the mobile device 3a and the server computer 4.
Receiving the broadcast data via a Bluetooth device on a second mobile device 3b, estimating a second receiving distance between the second mobile device 3b and the drug delivery device 1 based on the reception of the broadcast data, and forwarding the second receiving distance to the server computer 4. Again, this estimate may be based on Bluetooth's RSSI or other suitable information. If, as shown, all devices are within range of a Bluetooth connection, the drug delivery data of the second drug delivery device 11 analogously are also received by both mobile devices 3a, 3b and forwarded to the server together with a measure for the estimated receiving distance between the mobile device and the drug delivery device. Only the information that two different drug delivery devices are in proximity to a mobile device is important for the assignment and can be used to secure the communication.
Receiving and recognizing as being identical the drug delivery data forwarded by the first and second mobile devices 3a, 3b on the server computer 4. For this purpose, the server computer 4 can recognize the sender of the drug delivery data from the identification sent with them or from any other part of the data.
Assigning, on the server computer 4, the drug delivery data of the drug delivery device 1 received from the first or second or both mobile devices 3a, 3b to the first or the second mobile device 3a, 3b by comparing the first and second receiving distances. After the assignment has been made, the confirmation is only sent to precisely one mobile device 3a or 3b; in the example of FIG. 5, this is the first mobile device 3a. Similarly, confirmation is sent to the second mobile device 3b if the receiving distances transmitted to the server 4 show that the second mobile device 3b is closer to the first drug delivery device 1. Also similarly, the drug delivery data from the drug delivery device 11 can be assigned to the nearest mobile device 3b.

FIG. 6 also shows that a drug delivery device 1 can also perceive, receive and analyze Bluetooth advertising channels for data packets from other communication partners in the vicinity. In the same way that confirmations are received from a mobile device 3, this can also take place for confirmations, data packets or other signals which are not intended for the drug delivery device 1 in question, but for example for a second drug delivery device 11. The drug delivery device 1 may recognize such a communication as such and derive therefrom the presence of a second drug delivery device 11 within the Bluetooth range. Such a presence may also be quantified, for example by estimating the receiving distance. In a further embodiment, the drug delivery device 1 adds the presence information to its drug delivery data during transmission via the Bluetooth channel 5. The mobile device 3 forwards this to the server 4, and the server 4 evaluates the presence information. In the same way that the server 4 uses the receiving distances of the various devices within the range of Bluetooth technology to assign mobile devices 3 and drug delivery data, the server 4 can use any quantified or non-quantified presence information to form or check assignments and thus increase the security of the communication.

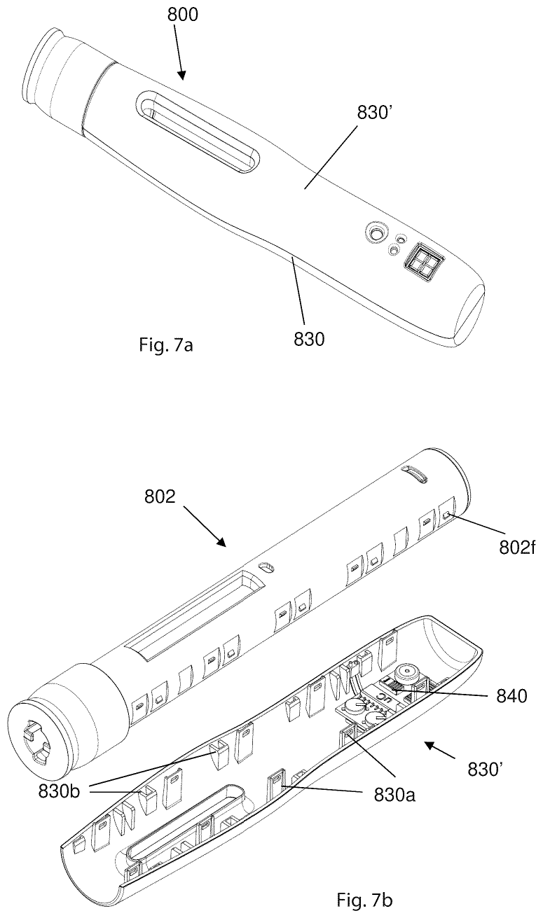

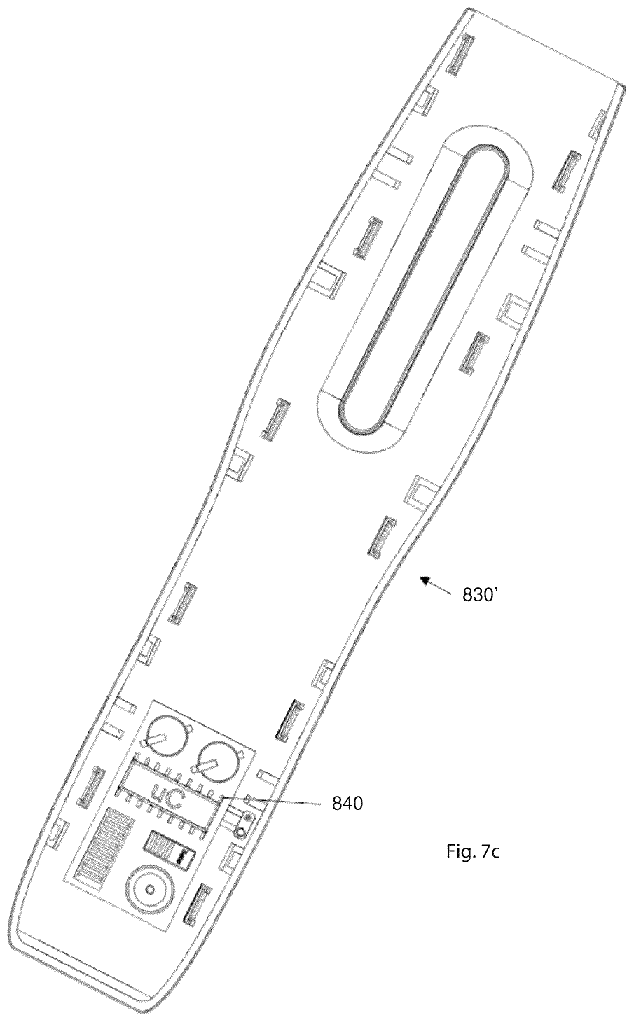

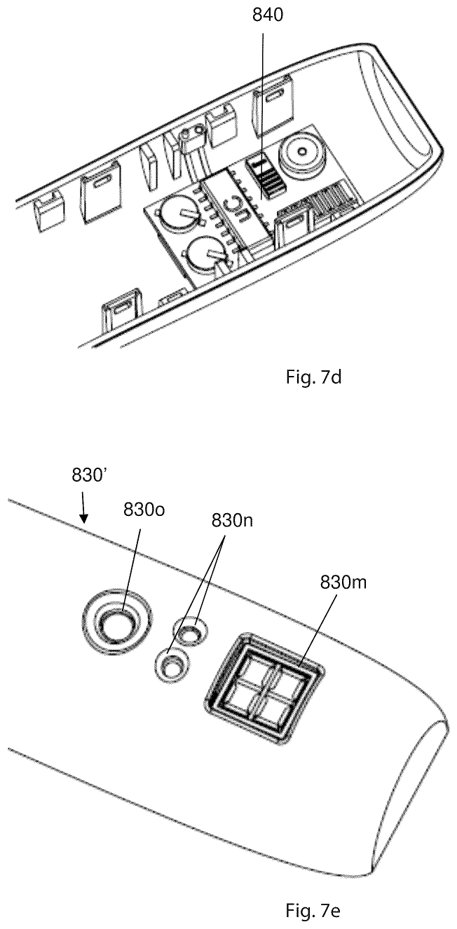

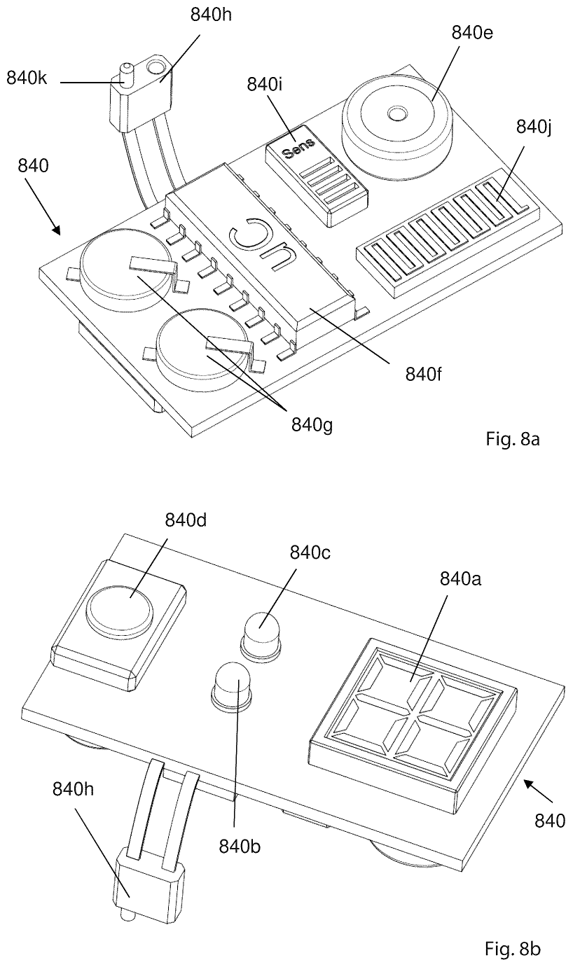

FIGS. 7a-7e and FIG. 8a-8b show an optional embodiment of an injection apparatus according to the invention (in the general description and further below also more commonly referred to as a drug delivery device). The injection apparatus 800 includes two housing-like shells 830 and 830′ (FIG. 7a), which can be attached to the housing 802 and together approximately cover the housing 802. The shell 830′ (FIGS. 7b, 7c, 7d, 7e) has the same shape as the shell 830 but has an additional electronic module 840 (FIG. 7d). The electronic module 840 is attached, for example glued or snapped, to the inside of the shell 830′. The shell 830′ has openings 830m, 830n and 830o through which elements of the electronic module are visible or accessible from the outside.

The injection apparatus 800 replaces the shown autoinjector 11 or the drug delivery device 1 from FIG. 1 and FIG. 2, and the shells 830, 830′ replace the add-on 12 from FIG. 3. The embodiments for the systems in FIG. 1 to FIG. 6 apply analogously to the present invention.

FIGS. 8a and 8b show the details of the electronic module 840 integrated into the shell 830′. FIG. 8a shows a first side of the electronic module. The microcontroller 840f is provided as a central control unit. This controls the additional components. The electronic module 840 thus includes a sensor module 840i, which can comprise temperature sensors, humidity sensors, light sensors, pressure sensors and/or a microphone, for example. Also provided on this side of the electronic module 840 are a loudspeaker or buzzer 840e as an acoustic emitter, and the communication module 840j for wireless communication with external devices. The power supply is likewise provided directly on the electronic module 840, for example in the form of batteries 840g. The rear side of the electronic module is shown in FIG. 8b. A pushbutton or button 840d, two LEDs 840b and 840c, and the display 840a are arranged there. The light-emitting diode 840b is designed as a green LED and the light-emitting diode 840c is designed as a red LED, although the color could also be different. The display 840a is designed as a simple 7-segment display having two digits. However, a higher resolution LCD or OLED display could also be implemented. The pushbutton 840d, the LEDs 840b and 840c and the display 840a are accessible or visible from the outside through the corresponding openings 830o, 830n and 830m in the shell 830′.

The sensor 840h is connected to the electronic module 840. As can be seen in FIGS. 8a and 8b, the sensor 840h includes a pin 840k. This pin 840k is movably mounted on the sensor 840h. If the shells 830 and 830′ are attached to the housing 802, the pin 840k is moved into the sensor 840h by a rib on the shell 830 and closes an electrical contact present in the sensor 840h. This contact closure is evaluated by the microcontroller 840f. In the present example, contact closure means that the injection apparatus, including a product container, is fully assembled, since the housing-like shells 830 and 830′ are the last parts which on the injection apparatus. As a result of the contact closure, the microcontroller activates an internal service life timer as well as the existing sensors 840i, such as a temperature sensor. Once it has elapsed, the service life timer signals when the injection apparatus should no longer be used because the expiry date of the medication in the product container has passed. The timer is therefore programmed during assembly specifically for the medication used and its shelf life. The signaling takes place optically via the red LED 840c, which may flash after expiry, and via the loudspeaker 840e, which may emit acoustic signals. The temperature sensor in the sensor 840i measures the temperature of the injection apparatus; the corresponding signal is evaluated by the microcontroller 840f and may be stored in an internal memory. If the temperature of the injection apparatus exceeds or falls below a predetermined value over a certain period of time and if it may then be assumed that the medication has been affected by the impact, the microcontroller activates LEDs, loudspeakers and/or the display to signal to the outside that the injection apparatus should no longer be used. The signals emitted differ from those which are emitted after the service life has elapsed.

In the present example, the sensor module 840i further includes a microphone with which the microcontroller 840f can determine when the injection apparatus 800 is used for an injection process. The triggering of the injection apparatus 800 and the associated movements in the dispensing mechanism emit a specific acoustic pattern which is recognized by the microcontroller 840f. The microcontroller 840f is now programmed such that it signals the injection to the user and then also signals to the user when the injection process has finished (and, for example, the injection needle can be removed from the tissue). In the present example, the green LED 840b begins to flash as soon as the microcontroller determines that the injection apparatus has been triggered; the optical display can thereby be supported by acoustic signals from the loudspeaker 840e (also by voice output such as: “The injection is in progress, please wait”). At the same time as the LED, the display 840a is activated and the intended injection duration is counted down in seconds in order to give the user an indication of how long the injection process will take. When the counter reaches zero, the injection process is complete. The green LED 840b no longer flashes, but continuously lights up as a sign of completion. Likewise, acoustic feedback can also be provided via the loudspeaker 840e, such as a voice output saying “injection process complete”. The microcontroller registers and saves the time and date of the injection process.

In addition to the parts of the electronic module 840 that have already been described in more detail, the electronic module can optionally comprise a communication module 840j which, by means of Bluetooth, WLAN or GSM, can send data from the injection apparatus 800 or can receive data or commands from the external environment. In the present example, the microcontroller 840f can send information by means of the communication module 840j via Bluetooth, for example in broadcast mode. For example, a nearby smartphone can receive the information. An associated smartphone app can then keep a record of a therapy. The unique identifier for the injection apparatus allows a conclusion to be drawn about the medication, the medication lot, the exact type of injection apparatus, and the production lot of the injection apparatus. The data collected by the app can then be transmitted to the treating physician, the insurer or to a server on the Internet.

The pushbutton 840d has two functions: on the one hand, a service life or function check can be carried out by briefly pressing the pushbutton 840d. If the pushbutton 840d is pressed briefly, the microcontroller 840f checks, on the one hand, whether the timer is running and, on the other hand, whether the sensor module 840i is delivering plausible measured values in which the microcontroller 840f compares the current measured values against a stored library of measured values. If both are the case, the microcontroller 840f issues a corresponding confirmation via the green LED 840b and the loudspeaker 840a. If one of the tests reveals a problem, the microcontroller provides feedback via the red LED 840c and the loudspeaker. A user can thus check at any time whether an injection apparatus can still be used or not.

### LIST OF REFERENCE SIGNS

1 Drug delivery device, patch injector (patch-type injection device)
1a Trigger button
1b Protective film patch
1c Reservoir viewing window
1d Status LED
3, 3a, 3b Mobile device, smartphone
4 Server, remote server, server computer or cloud
5 Bluetooth transmission or broadcast channel, unidirectional or bidirectional
6 Network and/or mobile communication/cell phone connection
7, 7a, 7b User or patient
11 Drug delivery device, autoinjector
11a Electronic module
12 Add-on
12a Opening
800 Injection apparatus/drug delivery device/autoinjector having shells
802 Housing
802f Attachment point
830, 830′ Shell
830a Holding device
830b Blocking device
830m Opening for display
830n Openings for LEDs
830o Opening for pushbutton
840 Electronic module
840a Display
840b, c LED
840d Button/pushbutton
840e Loudspeaker/buzzer
840f Microcontroller
840g Battery/batteries
840h Sensor
840i Sensor
840j Communication module
840k Pin

1. A method for transmitting drug delivery data for dose administration from a reservoir of a drug delivery device, wherein the method comprises the following steps:
encrypting the drug delivery data using a key on a processor of the drug delivery device;
wirelessly transmitting the encrypted drug delivery data together with a unique identifier for the drug delivery device in a transmission mode as transmission data via a Bluetooth unit of the drug delivery device and a Bluetooth transmission channel;
receiving, by a mobile device, the transmission data and the unique identifier for the drug delivery device;
forwarding, via a network interface of the mobile device, the transmission data and the unique identifier for the drug delivery device to a remote server;
receiving and decrypting, by the remote server, the encrypted drug delivery data in the transmission data using a matching key stored on the remote server, wherein the matching key is identified using the unique identifier; and
sending, using the remote server, a confirmation of receipt to the mobile device specifying the unique identifier for the drug delivery device.

2. The method according to claim 1, wherein the key of the drug delivery device and the matching key of the remote server form an asymmetric key pair, and the drug delivery data encrypted on the drug delivery device is configured to be decrypted only with the matching key of the remote server.

3. The method according to claim 1, wherein the mobile device forwards the encrypted drug delivery data unchanged and not decrypted.

4. The method according to claim 1, wherein no Bluetooth pairing takes place between the drug delivery device and the mobile device before the transmitting and receiving steps.

5. The method according to claim 1, wherein a user manually confirms on the mobile device that the dose has been administered.

6. The method according to claim 1, wherein the method further comprises the following steps:
receiving, by the mobile device, the confirmation of receipt;
forwarding, by the mobile device, the confirmation of receipt to the drug delivery device via the Bluetooth transmission channel; and
receiving, by the drug delivery device, the confirmation of receipt.

7. The method according to claim 1, further comprising the following steps:
receiving the transmission data via a Bluetooth unit on a first mobile device, estimating a first receiving distance between the first mobile device and the drug delivery device based on the received the transmission data, and forwarding the first receiving distance to the remote server;
receiving the transmission data from the drug delivery device via a Bluetooth unit on a second mobile device, estimating a second receiving distance between the second mobile device and the drug delivery device based on the received the transmission data, and forwarding the second receiving distance to the remote server;
receiving and recognizing as being identical the drug delivery data received from the first or second or both mobile devices on the remote server; and
assigning, on the remote server, the drug delivery data received from the first or second or both mobile devices to the first or the second mobile device by comparing the first and second receiving distances.

8. The method according to claim 7, wherein the estimating of the first or second receiving distance is performed by means of Bluetooth Received Signal Strength Indication (RSSI).

9. The method according to claim 7, wherein the drug delivery device is a first drug delivery device and is configured to recognize communication with a second drug delivery device in a Bluetooth transmission channel and derives therefrom a presence of the second drug delivery device within a Bluetooth range.

10. The method according to claim 9, wherein the first drug delivery device adds presence information to self-sent drug delivery data indicating the presence of the second drug delivery device.

11. The method according to claim 10, wherein the presence information contains an estimation of how close the second drug delivery device or at least one of the mobile devices is to the first drug delivery device.

12. The method according to claim 10, wherein the received distance estimations for the first and the second drug delivery devices are used by the remote server to determine an assignment of the first or the second drug delivery devices to the first or the second mobile device.

13. The method according to claim 9, wherein the first drug delivery device receives and analyzes confirmations of receipt via the Bluetooth transmission channel, wherein the confirmations of receipt are for the second drug delivery device.

14. The method according to claim 13, wherein the first drug delivery device adds presence information to self-sent drug delivery data indicating the presence of the second drug delivery device.

15. The method according to claim 14, wherein the presence information contains an estimation of how close the second drug delivery device or at least one of the mobile devices is to the first drug delivery device.

16. The method according to claim 14, wherein the received distance estimations for the first and the second drug delivery devices are used by the remote server to determine an assignment of the first or the second drug delivery devices to the first or the second mobile device.

17. An injection system, comprising:
a drug delivery device including a drive for the subcutaneous delivery of at least one dose of a fluid medication from a reservoir;
an electronic module comprising sensors for determining delivery processes, processing electronics for evaluating data from the sensors, and a wireless Bluetooth transmitter for wireless transmission of information; and
a memory configured to store a key that uniquely identifies the reservoir and which can be used to encrypt the wirelessly transmitted information,
wherein the Bluetooth transmitter is configured such that the encrypted information can be repeatedly transmitted wirelessly in transmission mode via a Bluetooth transmission channel together with a non-encrypted, unique identifier for the reservoir.

18. The injection system according to claim 17, further comprising a mobile device and a remote server configured to allow the mobile device to wirelessly forward encrypted drug delivery data unchanged and not decrypted, to the remote server.
